(19) United States
(12) Patent Application Publication
(10) Pub. No.: US 2026/0021152 A1
(43) Pub. Date: Jan. 22, 2026

(54) USE OF MALE FLOWER EXTRACT OF EUCOMMIA ULMOIDES OLIVER IN PREPARATION OF FOOD OR MEDICAMENT FOR RESISTING MUSCLE AGING

(71) Applicant: ZHEJIANG UNIVERSITY, HANGZHOU, Zhejiang Province (CN)

(72) Inventors: BAIYI LU, HANGZHOU, ZHEJIANG PROVINCE (CN); QI CHEN, HANGZHOU, ZHEJIANG PROVINCE (CN); WEISU HUANG, HANGZHOU, ZHEJIANG PROVINCE (CN); LIPENG WU, HANGZHOU, ZHEJIANG PROVINCE (CN); YUHANG ZHU, HANGZHOU, ZHEJIANG PROVINCE (CN); YIXUAN WANG, HANGZHOU, ZHEJIANG PROVINCE (CN)

(21) Appl. No.: 18/995,658

(22) PCT Filed: Jun. 6, 2023
(86) PCT No.: PCT/CN2023/098531

§ 371 Date: Jan. 16, 2025

(30) Foreign Application Priority Data
Jul. 26, 2022 (CN) 202210883992.1

(51) Int. Cl. A61K 36/46; A61K 31/7048; A61P 21/00

(52) U.S. Cl. CPC A61K 36/46; A61K 31/7048; A61P 21/00; A61K 2236/333; A61K 2236/39; A61K 2236/51; A61K 2236/53; A61K 2236/55

(57) ABSTRACT

The present invention discloses use of a male flower extract of Eucommia ulmoides Oliver in preparation of a food or a medicament for resisting muscle aging, belonging to the technical field of plant extracts. A main active ingredient of the male flower extract of Eucommia ulmoides Oliver is iridoids, and the composition of the iridoids includes aucubin, geniposide, geniposidic acid and asperuloside. The present invention reports for the first time that the male flower extract of Eucommia ulmoides Oliver and the iridoids contained therein have mitophagy activation activity and maintain normal mitochondrial function by effectively stimulating mitophagy. The extract has a relatively strong ability to prevent or alleviate aging-related muscle hypofunction, and has great application prospects in food, pharmaceutical and other fields.

### FIELD OF TECHNOLOGY

The present invention relates to the technical field of plant extracts, and specifically to use of a male flower extract of Eucommia ulmoides Oliver containing an iridoid ingredient in preparation of a food (including a pet food) or a medicament for resisting muscle aging.

### BACKGROUND TECHNOLOGY

Skeletal muscle is the power of the human motor system, and its aging is an important sign of body aging. As the most abundant plastic organ in vertebrates, it plays an important role in metabolism, movement, respiration, protection, daily physical activities, as well as maintaining posture and balance. In 2016, sarcopenia (characterized by progressive and generalized loss of skeletal muscle mass and function) was officially recognized as a disease, with the code ICD-10-CM (M62.84), which has drawn additional attention to this age-related disease. Aging-related muscle function decline is an important feature of the occurrence and development of sarcopenia.

Sarcopenia, especially aging-related muscle function decline, is closely associated with mitochondrial dysfunction in skeletal muscle. Clearance of dysfunctional mitochondria to maintain mitochondrial homeostasis in the aging process is mainly carried out through mitochondria-specific autophagy, namely mitophagy. However, the activity of mitophagy will decrease in the muscle aging process. All of these indicate the importance of mitophagy in maintaining muscle function in the aging process. In recent years, studies have shown that enhancement of mitophagy by administering single natural compounds (e.g., urolithin A, spermidine, tomatine) has beneficial effects on aging skeletal muscle, heart and neurons (Urolithin A induces mitophagy and prolongs lifespan in C. elegans and increases muscle function in rodents, Nat Med 2016; 22:879-88. Mitophagy inhibits amyloid-β and tau pathology and reverses cognitive deficits in models of Alzheimer's disease, Nat Neurosci 2019; 22:401-12. Cardioprotection and lifespan extension by the natural polyamine spermidine, Nat Med 2016; 22:1428-38. Tomatidine enhances lifespan and healthspan in C. elegans through mitophagy induction via the SKN-1/Nrf2 pathway, Sci. Rep 2017; 7:46208.). These studies have suggested that mitophagy enhancers may provide new options for preventing and alleviating aging muscle hypofunction.

Natural products, especially phytochemicals, are potential sources of active compounds for mitophagy regulation and anti-muscle aging. In recent years, edible flowers have become one of the new trends in the consumption of food resources and the development of plant-derived natural products. Notably, edible flower resources have potential value in delaying aging and related diseases. As a new food raw material approved by the Ministry of Health of China, the male flowers of Eucommia ulmoides Oliver have physiological activities such as lowering blood lipid, anti-obesity, anti-oxidation and anti-fatigue. As a main functionally active ingredient of the male flowers of Eucommia ulmoides Oliver, iridoids are widely distributed in traditional Chinese medicine, including plants in the Eucommiaceae family, the Rubiaceae family, etc. Iridoids have anti-inflammatory, anti-obesity, antibacterial, anti-tumor, neuroprotective and other pharmacological effects, and are also an important class of quality control ingredients in traditional Chinese medicine. However, the preventive and alleviating effects of the male flowers of Eucommia ulmoides Oliver and their functionally active ingredients on muscle aging and related hypofunction have not been reported.

### SUMMARY OF THE INVENTION

The object of the present invention is to explore the new functions of the male flowers of Eucommia ulmoides Oliver in preventing and alleviating the aging of skeletal muscle, and to extract the effective ingredients in the male flowers of Eucommia ulmoides Oliver, so as to fill the current research gaps regarding the above efficacy and functional ingredients of the male flower extract of Eucommia ulmoides Oliver.

In order to achieve the above object, the present invention adopts the following technical solutions:

In the present invention, Caenorhabditis elegans is used to screen 30 common domestic edible flowers in China, and it is found that the male flower extract of Eucommia ulmoides Oliver effectively prolongs the healthy lifespan of the nematodes, and especially improves the age-related muscle function decline. Qualitative and quantitative analyses by high-resolution mass spectrometry show that the main anti-aging active ingredient in the above male flower extract of Eucommia ulmoides Oliver is iridoids, including asperuloside, aucubin, geniposide and geniposidic acid. Using human-derived mt-Keima HEK 293T cells in vitro, these iridoids are found to have mitophagy-activating activity. Further, the preventive and alleviating effects of male flower extract of Eucommia ulmoides Oliver on aging-related muscle hypofunction are confirmed in mammalian models.

Therefore, the present invention provides use of a male flower extract of Eucommia ulmoides Oliver in preparation of a food or a medicament for preventing or alleviating aging-related muscle hypofunction, wherein an active ingredient in the male flower extract of Eucommia ulmoides Oliver includes iridoids.

The food includes a human food and a pet food.

The male flower extract of Eucommia ulmoides Oliver is an extract with iridoids as the main active ingredient obtained after extracting a male flower of Eucommia ulmoides Oliver by using an aqueous solution of an organic solvent or a pure aqueous solution. Specifically, a temperature of the extracting is 20-65° C.

Further, the iridoids include one or more of aucubin, geniposide, geniposidic acid and asperuloside.

The preventing or alleviating aging-related muscle hypofunction includes improving skeletal muscle mass, skeletal muscle strength, mitochondrial function and body motor ability. In present invention, the efficacy of the male flower extract of Eucommia ulmoides Oliver is studied through in vivo experiments on nematodes and mice. The extract can improve skeletal muscle mass, strength, mitochondrial function and body motor ability, etc., indicating that the extract has anti-muscle aging effects such as an effect of preventing or alleviating aging-related muscle hypofunction.

The aging-related muscle hypofunction is closely related to mitochondrial dysfunction in skeletal muscle, and related diseases include sarcopenia.

Further, the male flower extract of Eucommia ulmoides Oliver improves the mitochondrial function to delay muscle hypofunction by activating mitophagy activity of muscle cells, promoting ATP (adenosine triphosphate) synthesis and/or maintaining normal mitochondrial morphology. The male flower extract of Eucommia ulmoides Oliver delays muscle aging by improving mitochondrial function in skeletal muscle.

In present invention, the mitophagy active ingredients in the male flower of Eucommia ulmoides Oliver are identified, and it is determined that the main mitophagy-enhancing active ingredient in the male flower extract of Eucommia ulmoides Oliver is iridoids, including asperuloside, aucubin, geniposide and geniposidic acid. In order to ensure that the extract contains the above active ingredient, in the present invention, the extraction method is specified under the guidance of the property that the iridoid ingredients of the male flower of Eucommia ulmoides Oliver are easily soluble in water.

A method for preparing the male flower extract of Eucommia ulmoides Oliver includes steps of:
(1) adding a dried product of a male flower of Eucommia ulmoides Oliver into an aqueous solution of an organic solvent with a volume ratio of 0%-90% at a solid-liquid ratio of 1 g:20 mL-60 mL, extracting at a condition of 20-65° C., and separating to obtain an extracted liquid; and
(2) removing the organic solvent in the extracted liquid, then separating to obtain an extract liquid after adding water for full reconstitution, and drying to prepare the male flower extract of Eucommia ulmoides Oliver.

The male flower of Eucommia ulmoides Oliver refer to a stamen of a male tree of Eucommia ulmoides Oliver.

The raw material adopted in present invention is a dried product of the male flower of Eucommia ulmoides Oliver, which refers to a kind of raw materials of a fresh male flower of Eucommia ulmoides Oliver or an ordinary sun-dried male flower of Eucommia ulmoides Oliver dried until the water content does not exceed 10%. The raw material can be subjected to pulverization pretreatment or can be not subjected to pulverization pretreatment. Preferably, pulverizing the dried product of the male flower of Eucommia ulmoides Oliver is conducive to dissolution of an active substance.

In step (1), by controlling the solid-liquid ratio, the temperature of the extracting and the water content of the raw material, a relatively high extraction rate is comprehensively realized.

Preferably, a water content of the dried product of the male flower of Eucommia ulmoides Oliver is ≤10%, and a solid-liquid ratio is 1 g:40 mL.

The extractant adopted in the present invention can be an aqueous solution containing an organic solvent, or can be pure water. Preferably, the organic solvent is ethanol, methanol or n-butanol, and a volume ratio concentration of the aqueous solution of the organic solvent is 65%-80%.

Preferably, a temperature of the extracting is 45-60° C., and time of the extracting is 20-30 hours.

Preferably, an extraction process is combined with ultrasonic treatment, and ultrasonic conditions are as follows: an ultrasonic frequency is 200-500 kHz, an ultrasonic intensity 5-15 Wcm², time is 1.5-3.0 hours, and a solution for the extracting is left to stand still after the ultrasonic treatment is completed.

In step (2), the organic solvent is removed by evaporation under reduced pressure to obtain the concentrated liquid, water with a volume 5-15 times that of the concentrated extracted liquid is added for full reconstitution, then the extracted liquid is filtered and separated to obtain the extract liquid, and then the extract liquid is freeze-dried to obtain the male flower extract of Eucommia ulmoides Oliver.

The active ingredients of the male flower extract of Eucommia ulmoides Oliver prepared by the above method are mainly asperuloside, aucubin, geniposide and geniposidic acid. A weight content of asperuloside is 0.05%-5.0%; a weight content of aucubin is 0.05%-10.0%; a weight content of geniposide is 0.1%-5.0%; and a weight content of geniposidic acid is 0.1%-10.0%. Considering that the male flower extract of Eucommia ulmoides Oliver and the main active ingredient thereof iridoids have anti-muscle aging efficacy such as efficacy of preventing and alleviating aging-related muscle dysfunction, they can be used for preparation of a related food and medication.

The food is a functional food or beverage having efficacy of preventing or alleviating aging-related muscle hypofunction. The male flower extract of Eucommia ulmoides Oliver or the main active ingredient thereof is prepared into the functional food or beverage in combination with a food-acceptable excipient or auxiliary ingredient.

The medicament is a pharmaceutical preparation having efficacy of preventing or alleviating aging-related muscle hypofunction. The male flower extract of Eucommia ulmoides Oliver or the main active ingredient thereof is prepared into the pharmaceutical preparation in combination with a pharmaceutically acceptable excipient or auxiliary ingredient. The preparation may be a tablet, a powder, a granule, a capsule, an oral liquid, a sustained-release agent, etc.

The present invention has the following beneficial effects:

(1) The present invention reports for the first time that the male flower extract of Eucommia ulmoides Oliver and asperuloside, aucubin, geniposide and geniposidic acid contained therein have mitophagy-activating activity, and these ingredients can maintain normal mitochondrial function by effectively stimulating mitophagy. The extract has a relatively strong ability to prevent or alleviate aging-related muscle hypofunction, and has great application prospects in human food, pet food, pharmaceutical and other fields.

(2) Through the technology of the present invention, the effective extraction of the ingredients with the efficacy of delaying muscle aging in the male flower of Eucommia ulmoides Oliver is achieved. The water content of the raw material and extraction conditions are strictly controlled to improve the yield of the extract. Further, after concentrating the extracted liquid, adding the reconstitution process helps dissolve the water-soluble iridoid ingredients in the male flower of Eucommia ulmoides Oliver and increase their contents. The obtained male flower extract of Eucommia ulmoides Oliver has good quality with a stable property, and simple process operation and low requirement for production equipment, which is conducive to industrialization development.

### BRIEF DESCRIPTION OF THE DRAWINGS

FIG. 1 is a total ion current chromatogram of a male flower extract of Eucommia ulmoides Oliver in Example 1, where A is an anion mode, and B is a cation mode.

FIG. 2 is a secondary mass spectrum of asperuloside in an extract, where A is an anion mode, and B is a cation mode.

FIG. 3 is a secondary mass spectrum of aucubin in an extract under an anion mode.

FIG. 4 is a secondary mass spectrum of geniposide in an extract under a cation mode.

FIG. 5 is a secondary mass spectrum of geniposidic acid in an extract, where A is an anion mode, and B is a cation mode.

FIG. 6 is a quantitative spectrum of iridoid ingredients in an extract, including 1-aucubin, 2-geniposide, 3-geniposidic acid and 4-asperuloside. A is a standard solution, and B is a sample solution of an extract.

FIG. 7 shows structural formulas of iridoid ingredients in an extract.

FIG. 8 is a diagram showing changes in a motion state of nematodes after being fed with a male flower extract of Eucommia ulmoides Oliver group and a vehicle group for 1, 5 and 9 days, where “Vehicle” represents the vehicle group, “EUFE” represents male flower extract of Eucommia ulmoides Oliver group, and the same applies below.

FIG. 9 is a diagram showing changes in an average speed of motion of nematodes after being fed with a male flower extract of Eucommia ulmoides Oliver group and a vehicle group for 1, 5 and 9 days.

FIG. 10 is a diagram showing morphological changes of body wall muscle fibers of nematodes after being fed with a male flower extract of Eucommia ulmoides Oliver group and a vehicle group for 1 and 5 days.

FIG. 11 is a diagram showing morphological changes of muscle mitochondria of nematodes after being fed with a male flower extract of Eucommia ulmoides Oliver group and a vehicle group for 9 days.

FIG. 12 is a diagram showing changes in muscle mitophagy activity of nematodes after being fed with a male flower extract of Eucommia ulmoides Oliver group, a vehicle group and a positive control group for 1 day.

FIG. 13 is a diagram showing in vitro activation of mitophagy by asperuloside.

FIG. 14 is a diagram comparing activity of iridoid ingredients in male flowers of Eucommia ulmoides Oliver in activating mitophagy, where Asp represents asperuloside, Auc represents aucubin, Gen represents geniposide, and Gen a represents geniposidic acid.

### DETAILED DESCRIPTION OF THE EMBODIMENTS

The present invention will be further described below in conjunction with specific embodiments. The following examples are merely used to illustrate the present invention and are not intended to limit the scope of application of the present invention. Without departing from the spirit and essence of the present invention, modifications or substitutions to the methods, steps or conditions of the present invention are all within the scope of the present invention.

The test methods used in the following examples are all conventional methods unless otherwise specified; and the materials, reagents, etc. used are, unless otherwise specified, all commercially available reagents and materials.

The raw material male flowers of Eucommia ulmoides Oliver used in the examples are purchased from Zhangjiajie, Hunan Province, and uniformly baked at 40° C. for more than 24 hours.

The iridoid monomer ingredients in male flowers of Eucommia ulmoides Oliver adopted in the quantitative analysis and activity examples, including asperuloside (CAS: 14259-45-1), aucubin (CAS: 479-98-1), geniposide (CAS: 24512-63-8) and geniposidic acid (CAS: 27741-01-1), are purchased from Chengdu HerbSubstance Pure Biotechnology Co., Ltd., and all have a purity of ≥ 98%.

### Example 1

1. Preparation of a Male Flower Extract of Eucommia ulmoides Oliver

Preparation method: 1 kg of dried stamens of male flowers of Eucommia ulmoides Oliver with a water content of ≤10% (determined according to the direct drying method in GB 5009.3-2016, and if the water content requirement was not met, the stamens were continued to be baked and dried at 40° C.) was added to an 80% (v/v) ethanol aqueous solution at a solid-liquid ratio of 1 g:40 mL. The temperature was controlled at 45° C., and extraction was carried out under an ultrasonic frequency of 400 kHz and ultrasonic intensity of 10 W/cm² for 2 hours for full extraction. The solution for the extracting was left to stand for 24 hours.

The extracted liquid and the precipitate were separated by centrifugation. Ethanol was recovered by evaporation under reduced pressure to obtain a concentrated liquid. After water with a volume 10 times that of the concentrated liquid was added, sufficient stirring was performed for reconstitution. The extract liquid and the insoluble substances were separated by centrifugation. The extract liquid was freeze-dried to obtain the male flower extract of Eucommia ulmoides Oliver.

### 2. Qualitative and Quantitative Analyses of the Obtained Extract

### 1) Qualitative Analysis of the Extract-UPLC-QE High-Resolution Mass Spectrometry

The ingredient qualitative analysis of the extract was performed by using the UPLC-QE high-resolution mass spectrometer. The determination conditions were as follows: the ACQUITY BEH C₁₈ column (2.1 mm×100 mm, ϕ 1.7 μm, Waters) was used for UPLC analysis, the column temperature was 40° C., the flow rate was 0.4 mL/min, and the mobile phase was a 0.1% aqueous formic acid solution (A) and a 0.1% formic acid-acetonitrile solution (B). The gradient procedure of the mobile phase was as follows: 0 min (A:B=95:5), 3 min (A:B=75:25), 4 min (A:B=35:65) and 10 min (A:B=35:65). The injection volume was 10 μL. QE operated in positive and negative ion mode. The operation parameters were set as follows: the cone voltage was 30 v, the capillary voltage was 2 kv, and the source temperature was 100° C. The data were recorded in a mass-to-charge ratio (m/z) range from 95 to 1400 with scan time of 0.25 second and a scan interval of 0.02 second for 10 minutes. On this basis, combined with the retrieval matching of mass spectrometry database Compound Discoverer™ and literature query, the possible ingredients were determined.

### 2) Quantitative Analysis of Iridoid Ingredients in the Extract-UPLC-PDA Chromatography

The quantitative analysis of iridoid ingredients in the extract was performed by a second-class HPLC chromatograph equipped with PDA detector. The determination conditions were as follows: the injection volume was 10 μL, the reversed-phase ODS-2 Hypersil C₁₈ column (4.6 nm×250 nm, ϕ 5 μm, Thermo Fisher Scientific) was used for HPLC analysis, the column temperature was 30° C., the flow rate was 1 mL/min, and the mobile phase was a 0.5% aqueous phosphoric acid solution (A) and methanol (B). The gradient procedure of the mobile phase was as follows: 0 min (A:B=95:5), 30 min (A:B=85:15) and 55 min (A:B=70:30).

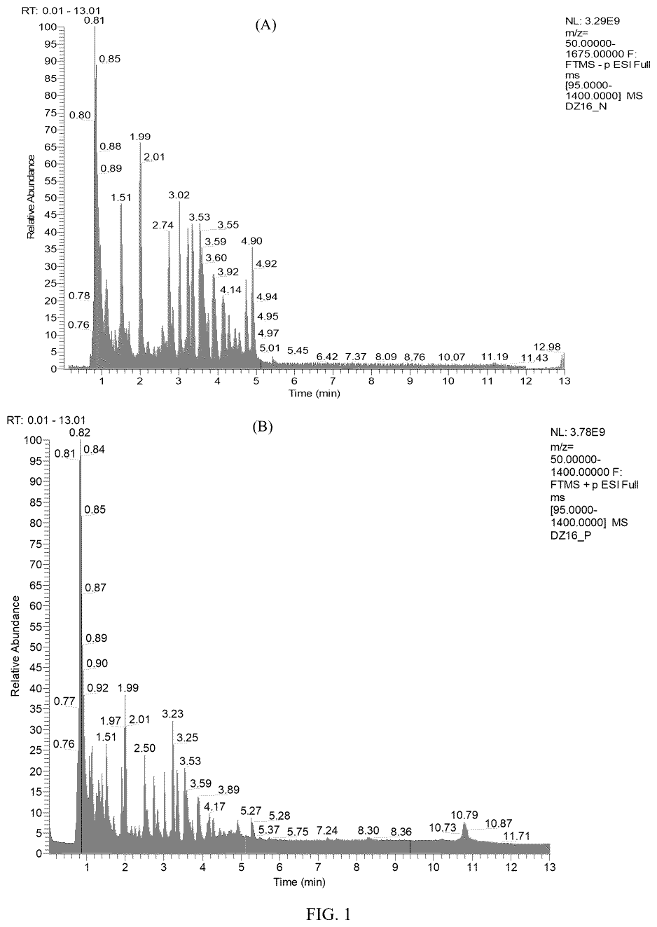

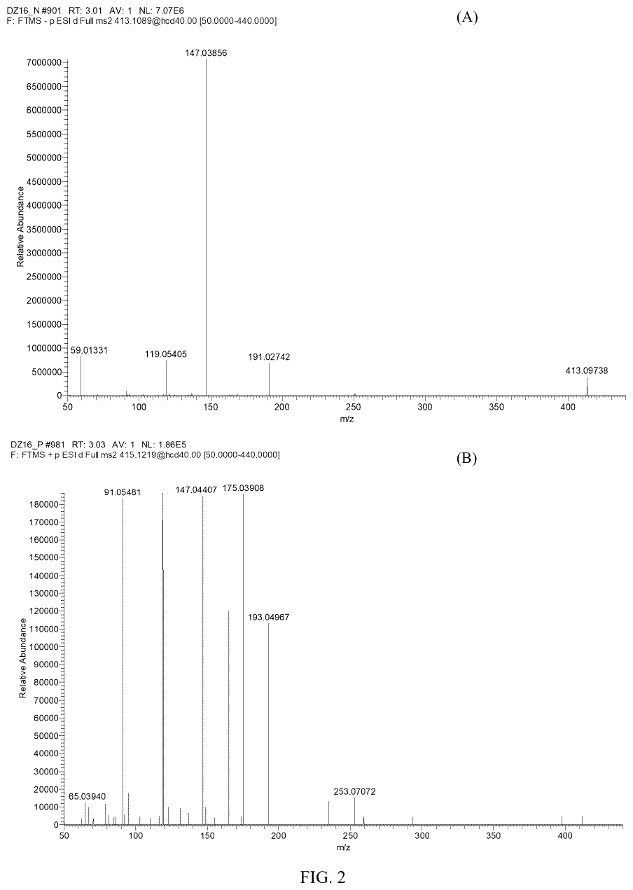

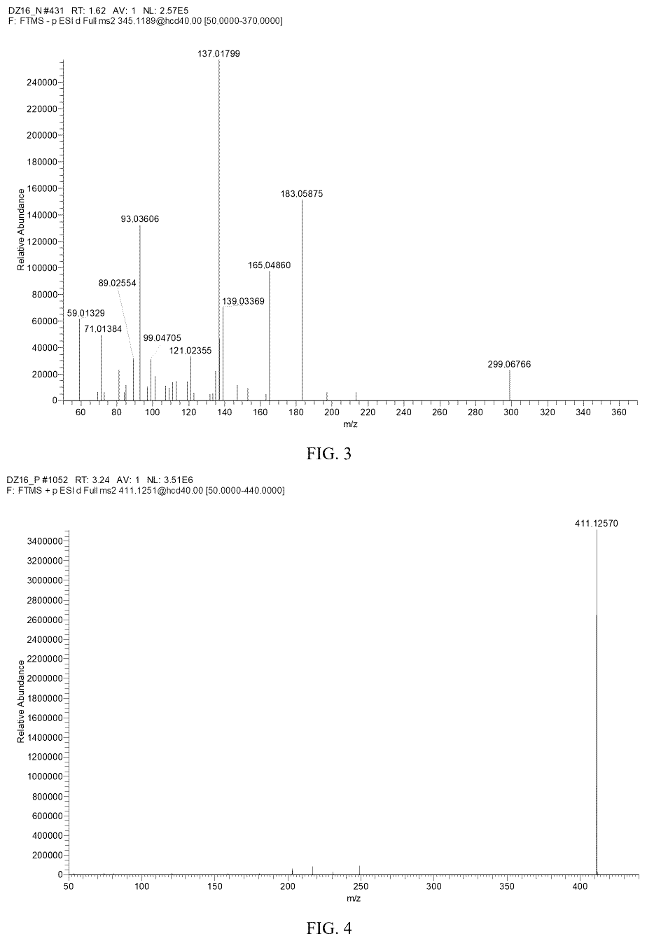

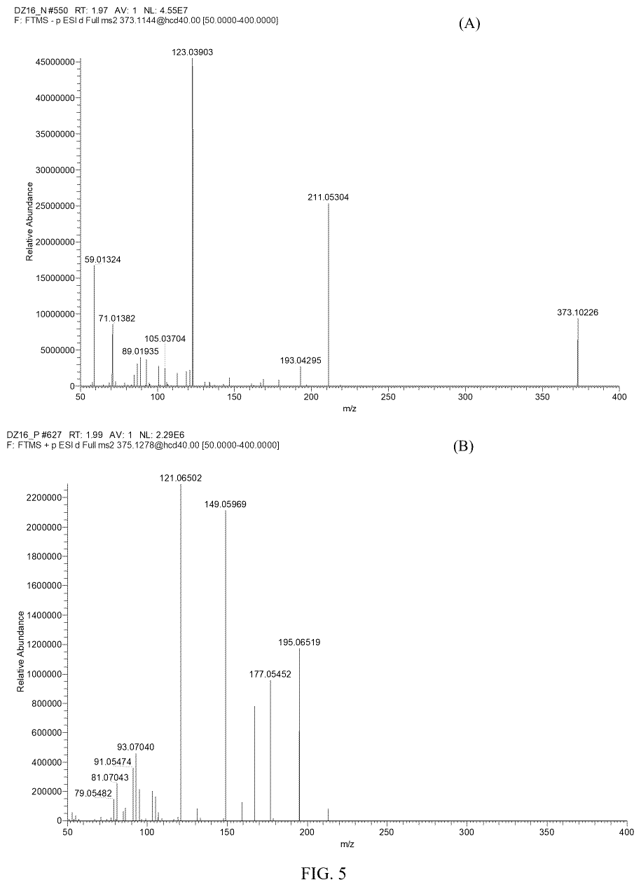

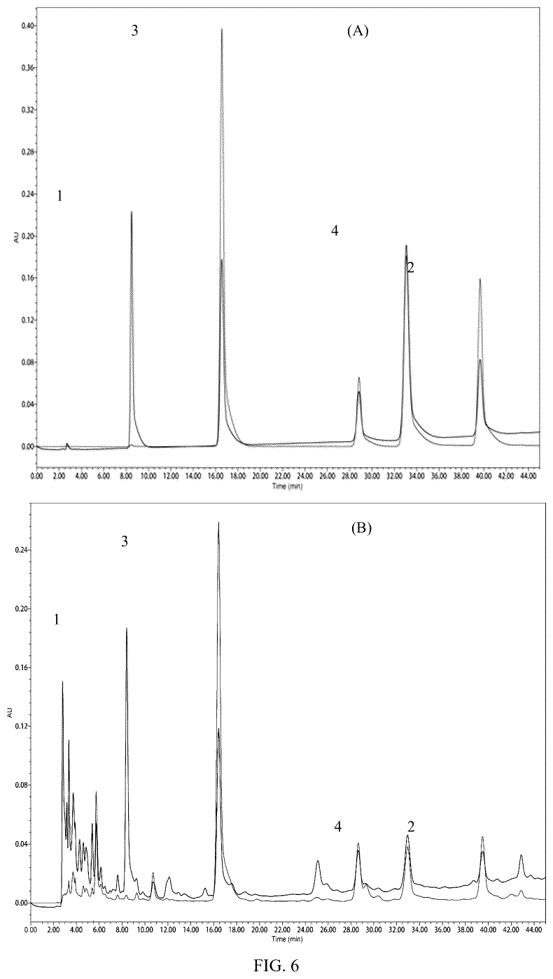

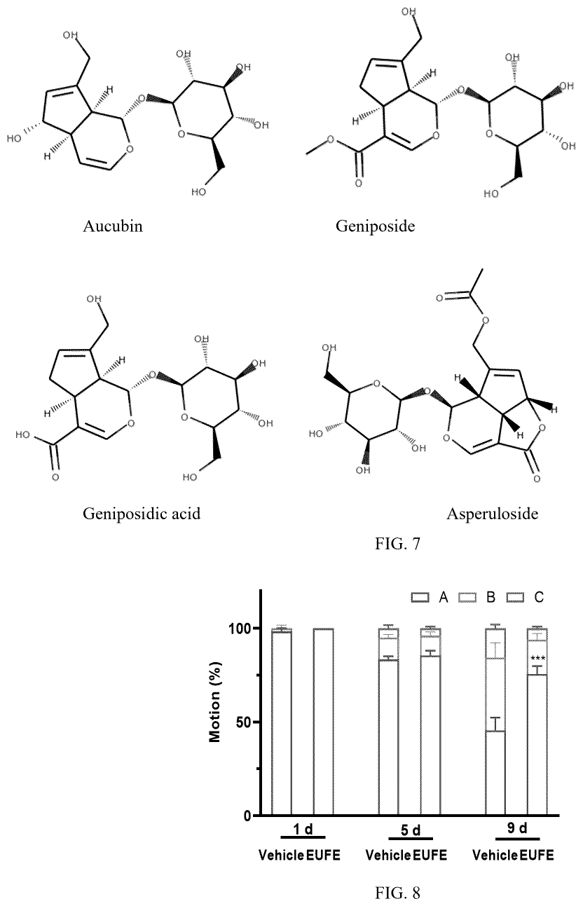

The above male flower extract of Eucommia ulmoides Oliver was subjected to qualitative analysis under the anion and cation modes using UHPLC-QE-MS. FIG. 1 is the corresponding total ion current chromatogram. A total of four iridoid ingredients in the male flower extract of Eucommia ulmoides Oliver were identified, including asperuloside, aucubin, geniposide, and geniposidic acid. Their secondary mass spectra are shown in FIGS. 2-5, respectively. The characteristic secondary mass spectrometry fragments of aucubin were only identified in the anion mode (FIG. 3), and the characteristic secondary mass spectrometry fragments of geniposide were only identified in the cation mode (FIG. 4). Further, the quantitative analysis of these four iridoid ingredients was performed by HPLC. The HPLC chromatograms of the standard solution and the extract sample solution are shown in FIG. 6A and FIG. 6B, respectively. It is to be emphasized that aucubin hardly had any absorption at 236 nm, and 206 nm was the appropriate ultraviolet absorption setting condition for its quantification. The results showed that geniposidic acid had the highest content, which was 6.39±0.33%, the content of aucubin was 6.05±0.22%, the content of geniposide was 1.85±0.16%, and asperuloside had the lowest content, which was 0.56±0.01%. The structural formulas of the above four iridoid ingredients are shown in FIG. 7.

### Activity Detection Example 1

Caenorhabditis elegans, a model organism, was fed with 100 μg/mL of the male flower extract of Eucommia ulmoides Oliver (abbreviated as EUFE) in Example 1. The changes in the motor ability and the morphology of body wall muscle fibers of the nematodes under natural aging conditions (on days 1, 5 and 9) were compared with those of a vehicle (water) control. The specific method was as follows:

1. motor ability: before the nematodes were transferred, their motion states were manually recorded as follows: moving autonomously, recorded as A, moving only after being touched, recorded as C, and the state between A and C, recorded as B. Then the nematodes in state A were picked onto a blank plate and video recordings were made. The average speed of motion was analyzed using WormLab. For each experiment, each group contained at least 30 worms. The motor ability of the nematodes was described more accurately by combining these two indicators.

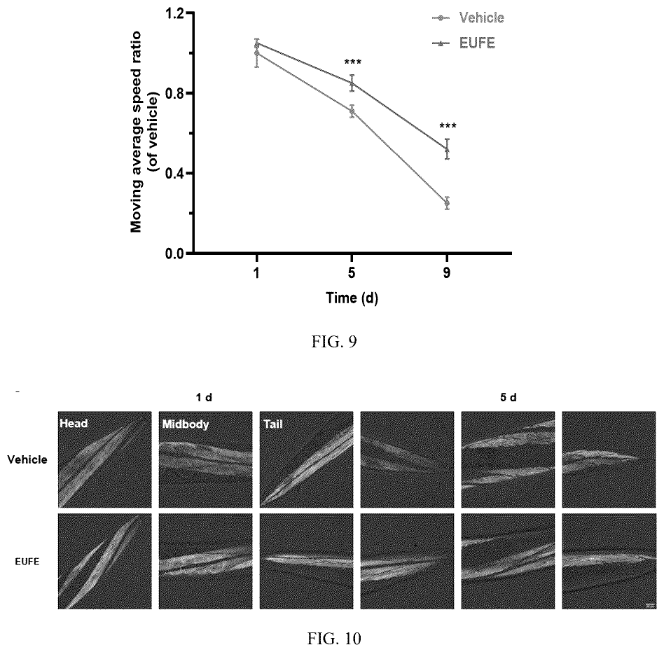

The results are shown in FIGS. 8 and 9. Compared with the vehicle, after being treated with EUFE for 9 days, a larger proportion of the nematode population (in state A) was able to move autonomously, and their average speed of motion was significantly faster.

2. morphology of body wall muscle fibers: the nematode RW1596 strain was used for the observation of the morphology of body wall muscle fibers under bright field (BF) and GFP channel (the excitation and emission wavelengths were 488 nm and 510-540 nm, respectively). The worms were pretreated with the vehicle or 100 μg/mL EUFE for 1 day and 5 days. Different groups of the nematodes were anesthetized using an M9 buffer solution containing 5 mM levamisole and then placed on 1% agarose gel on a thin glass slide. The nematode heads, midbodies and tails were photographed respectively using a Zeiss LSM 880 confocal microscope equipped with a 40× water immersion objective.

The results are shown in FIG. 10. Regarding the morphology of body wall muscle fibers, on the 1st day, the body wall muscle fiber tissues at the heads, midbodies and tails of the nematodes in both groups were intact and arranged in an orderly manner. However, on the 5th day, as indicated by the red arrows, the nematodes in the vehicle group showed obvious fractures, deletions or disordered arrangements at the head, midbody and tail. In contrast, the muscle morphology of the nematodes in the EUFE-treated group remained in a relatively good condition.

### Activity Detection Example 2

Study on the effect of feeding with the male flower extract of Eucommia ulmoides Oliver on the mitochondrial health and mitophagy activity in the body wall muscle cells of nematodes. The specific method was as follows:

1. mitochondrial morphology: wild-type nematodes were treated with a vehicle or EUFE for 9 days. After collection and cleaning, samples for transmission electron microscopy were prepared. The nematodes were fixed overnight using 2.5% glutaraldehyde. After fixation, the samples were rinsed with a 0.1 M phosphate buffer solution at pH 7.4. Post-fixation was carried out with 1% osmium tetroxide for 1-2 hours, followed by rinsing with the buffer solution again. Then the samples were dehydrated using ethanol with a series of gradient concentrations and acetone. They were then immersed in a mixture of acetone and Spurr embedding medium and finally embedded in 100% Spurr embedding medium overnight. The samples were cured at 70° C. for 36 hours. Ultra-thin sections (70-90 nm) were made using an ultra-microtome and transferred onto a 200-mesh copper grid. The grid was stained with lead citrate and uranyl acetate (a saturated solution in 50% (v/v) ethanol). The sections were observed by a Hitachi H-7650 transmission electron microscope.

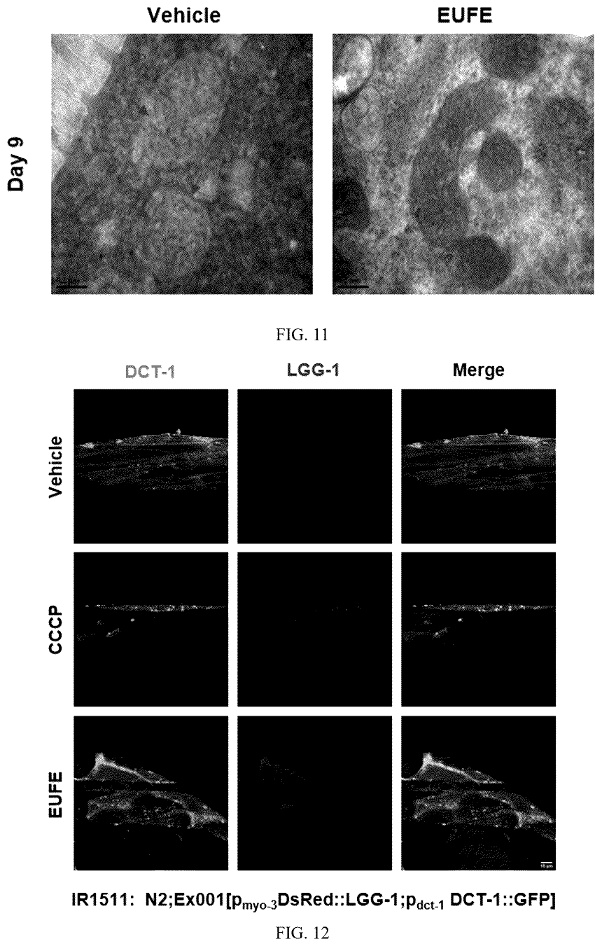

The results are shown in FIG. 11. After the nematodes in the vehicle group grew for 9 days, defects appeared in the outer membrane of their muscle mitochondria, and the mitochondrial cristae were reduced and became blurred. In contrast, treatment with EUFE maintained the mitochondrial morphology well.

2. mitophagy activity: the nematode IR1511 strain was used for mitophagy detection. This strain expresses GFP-labeled DCT-1 and DsRed-labeled LGG-1 in body wall muscles. The co-localization of GFP and DsRed indicates the fusion of mitochondria with lysosomes, i.e., the occurrence of mitophagy. DsRed was excited with a 561 nm laser. Both the vehicle and EUFE were used for treatment for 1 day, and CCCP (carbonyl cyanide ester-3-chlorophenylhydrazone), an inducer of mitophagy, was used as a positive control. A Zeiss LSM 880 confocal microscope equipped with an 83× oil immersion objective was used for photographing.

By using the IR 1511 strain, as shown in FIG. 12, consistent with the mitophagy inducer CCCP, treatment with EUFE for 1 day increased the expression of LGG-1 in the body wall muscles of the nematodes. More importantly, EUFE increased the co-localization of LGG-1 and DCT-1, indicating enhanced mitophagy activity. DCT-1 (a homolog of NIX/BNIP3L) is located on the outer membrane of mitochondria and is a key regulator of mitophagy.

### Activity Detection Example 3

Study on the in vitro activation of mitophagy activity by iridoid ingredients in the male flowers of Eucommia ulmoides Oliver, including asperuloside, aucubin, geniposide and geniposidic acid. The specific method was as follows:

293T cells were cultured and passaged in the normal way, and inoculated in a 24-well plate, with 0.5 mL inoculated in each well. The cell density was 5×10⁴ cells/mL, and a total of 2.5×10⁴ cells were inoculated. Before inoculation, the cells in the centrifuge tube were pipetted evenly to ensure that the cell density at the beginning and at the end was consistent. After standing for 5-10 minutes, the plate was gently placed in the incubator. After overnight incubation, the original complete medium was removed, and 0.3 mL of a complete medium solution containing 1.5×10⁵ TU lentiviruses was added to each well. After incubation at 37° C. for 8 hours, 200 μL of the complete medium was added to each well. After 24 hours of transfection, the medium was replaced until the cell density reached 80-90%, and the cells were digested and passaged a T25 flask. After the cells grew to confluence, the transfection efficiency was observed using a fluorescence microscope. The successfully transfected cells were screened using a complete medium containing 0.5 μg/mL of a puromycin hydrochloride solution, and maintained with 0.25 μg/mL of the solution. Thus, mt-Keima cells were obtained.

The confocal dishes were inoculated with 1 mL per dish, with a cell density of 2×10⁵ cells/mL. After standing for 5-10 minutes, the dishes were gently placed in the incubator. When the cells grew to 30-40%, the drug was administered. After treating mt-Keima cells with 10 μM different iridoid monomer ingredients from the male flowers of Eucommia ulmoides Oliver or 10 μM CCCP for 6 hours, the medium was replaced with a fresh complete medium, and then live-cell confocal observation was performed directly. Confocal parameters: the excitation wavelengths were 440 nm and 596 nm each, and the emission wavelength was 620 nm, observed by a 40× water immersion objective.

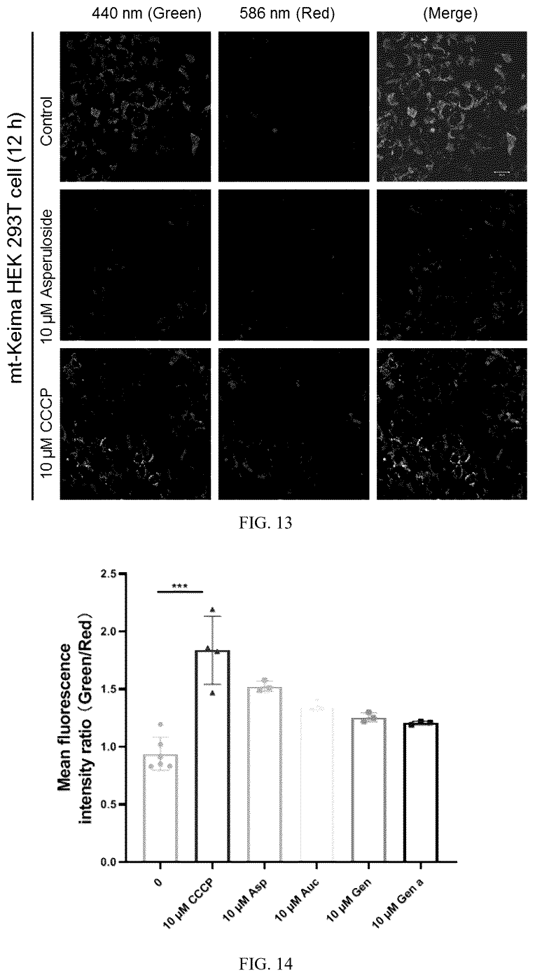

The results are shown in FIG. 13 and FIG. 14. Treatment with the positive drug CCCP significantly induced the occurrence of mitophagy in the 293T cells. The monomer ingredient represented by asperuloside in the male flowers of Eucommia ulmoides Oliver also had mitophagy-activating activity. Moreover, through the comparison of the average fluorescence density ratio, the effect of asperuloside was better than that of the other three monomer ingredients at the same concentration. Combined with the fact that the male flower extract of Eucommia ulmoides Oliver may improve mitochondrial function by activating mitophagy activity to delay muscle aging, the above iridoid ingredients with mitophagy-activating activity were the main active ingredients for the male flower extract of Eucommia ulmoides Oliver to exert the efficacy of preventing or alleviating muscle aging.

### Activity Detection Example 4

Study on the activity of the male flower extract of Eucommia ulmoides Oliver fed to 18-month-old ICR elderly male mice to delay their muscle hypofunction. The specific method was as follows:

Eighteen-month-old ICR male elderly mice purchased from the Animal Research Center of East China Normal University were housed singly in cages, and 5-6-month-old BALB/c male young mice were housed in groups in the specific pathogen-free animal facility of Zhejiang Chinese Medical University. During this period, the mice were allowed to eat and drink freely. The dark/light cycle in the animal facility was 12 hours, and the temperature and humidity were maintained at 23±2° C. and 50%±5%, respectively. All the procedures and protocols in this experiment were approved by the Animal Ethics Committee of Zhejiang Chinese Medical University according to the standards.

After two weeks of adaptation, the mice were divided into the following five groups, including the young mice group, the control elderly group, and the low-, medium-, and high-dose treatment groups of the male flower extract of Eucommia ulmoides Oliver (administered by gavage at 25, 50 and 100 mg/kg/d, respectively), with 8 mice in each group. The young mice group and the control group were gavaged with an equal volume of normal saline. Except the young mice group which was fed with normal standard feed, the other four groups of mice were fed with 42% high-fat feed.

1) Skeletal muscle mass: after 3 months of intervention for each group of mice, the mice were sacrificed. Then, four muscles in the left hind limb of each group of mice, including the gastrocnemius (GA), soleus (SOL), tibialis anterior (TA) and extensor digitorum longus (EDL), were carefully excised and weighed respectively.

2) Forelimb grip strength: in the intervention process, the mice were trained to grasp the horizontal bar of the grip strength meter every month, and the bar was gently pulled backward until the mice could no longer resist the pulling force and released the bar. The force was recorded by a dynamometer.

3) Exhaustive running time: the mice were trained to run adaptively on a treadmill at a speed of 10 m/min and an incline angle of 0° for two days. On the third day, an exhaustive running test was conducted. It started at a speed of 5 m/min with an incline angle of 0°, and then both the speed and the incline angle were increased by 5 m/min and 5° every 5 minutes respectively until reaching 20 m/min and 14° respectively. When the mice did not return to the track for more than 20 seconds and showed a significantly weakened response to external stimuli, the exhaustive running time was recorded.

The results are shown in Tables 1-3.

TABLE 1
Effect of the male flower extract of Eucommia ulmoides Oliver
on changes in skeletal muscle mass of naturally aging mice (n = 8)

| | | SOL | | EDL |
|---|---|---|---|---|
| Group | GA (mg) | (mg) | TA (mg) | (mg) |
| Young group | 211.4 ± 5.2*** | 8.0 ± 0.7 | 65.8 ± 4.3*** | 13.0 ± 0.8 |
| Elderly | 169.2 ± 2.3 | 7.2 ± 0.5 | 52.5 ± 1.8 | 12.4 ± 0.6 |
| control | | | | |
| group | | | | |
| Example 1 | 180.3 ± 3.1^{###} | 7.4 ± 0.5 | 55.4 ± 2.2^{#} | 12.4 ± 0.3 |
| Low-dose | | | | |
| group | | | | |
| Example 1 | 191.5 ± 3.0^{###} | 7.5 ± 0.6 | 58.3 ± 1.3^{###} | 12.7 ± 0.5 |
| Medium- | | | | |
| dose group | | | | |
| Example 1 | 196.0 ± 2.8^{###} | 7.6 ± 0.3 | 61.2 ± 3.0^{###} | 12.9 ± 0.6 |
| High-dose | | | | |
| group | | | | |

Note:
*indicates the comparison between the elderly control group and the young group,
*represents P < 0.05,
**represents P < 0.01, and
***represents P < 0.001;
^{#}indicates the comparison between the low-, medium-, and high-dose groups of Example 1 and the elderly control group,
^{#}represents P < 0.05,
^{##}represents P < 0.01, and
^{###}represents P < 0.001;
and the same applies below.

TABLE 2
Effect of the male flower extract of Eucommia ulmoides Oliver on
changes in forelimb grip strength of naturally aging mice (n = 8)

| | Average maximum | Average maximum |
|---|---|---|
| | forelimb grip | forelimb grip strength |
| Group | strength (N)-initial | (N)-after three months |
| Young group | 4.1 ± 0.9*** | 4.2 ± 1.0*** |
| Elderly control | 2.5 ± 0.2 | 1.3 ± 0.5 |
| group | | |
| Example 1 | 2.4 ± 0.3 | 1.5 ± 0.1 |
| Low-dose group | | |
| Example 1 | 2.5 ± 0.4 | 2.0 ± 0.3^{##} |
| Medium-dose | | |
| group | | |
| Example 1 | 2.6 ± 0.2 | 2.2 ± 0.3^{###} |
| High-dose group | | |

TABLE 3
Effect of the male flower extract of Eucommia ulmoides Oliver on
changes in exhaustive running time of naturally aging mice (n = 8)

| | Average exhaustive | Average exhaustive |
|---|---|---|
| | running time | running time (min)-after |
| Group | (min)-initial | three months |
| Young group | 90.5 ± 1.9*** | 91.8 ± 3.1*** |
| Elderly | 70.2 ± 2.1 | 50.5 ± 1.8 |
| control group | | |
| Example 1 | 68.9 ± 1.5 | 54.8 ± 1.2^{###} |
| Low-dose | | |
| group | | |
| Example 1 | 69.4 ± 1.6 | 60.0 ± 0.9^{###} |
| Medium- | | |
| dose group | | |
| Example 1 | 68.4 ± 2.2 | 61.2 ± 1.1^{###} |
| High-dose | | |
| group | | |

As shown in Table 1, the soleus (SOL) and extensor digitorum longus (EDL) were less affected by aging and a high-fat diet, while the gastrocnemius (GA) and tibialis anterior (TA) were more affected by aging and a high-fat diet. The elderly mice showed a tendency of mass reduction. The intervention with the male flower extract of Eucommia ulmoides Oliver in Example 1 prevented the mass reduction of the above muscle types and exhibited dose-dependence. As shown in Table 2 and Table 3, in addition to skeletal muscle mass, skeletal muscle function also declined with aging, which was manifested specifically by a significant decrease in forelimb grip strength and exhaustive running time after three months of high-fat diet intervention in the aging mice. However, the interventions with low-, medium-, and high-dose male flower extract of Eucommia ulmoides Oliver in Example 1 could all prevent the decline of the above functions of skeletal muscles. Therefore, the male flower extract of Eucommia ulmoides Oliver had the efficacy of preventing aging-related muscle hypofunction.

### Activity Detection Example 5

Study on the effect of the male flower extract of Eucommia ulmoides Oliver on the muscle ATP content in 18-month-old ICR elderly mice. The specific method was as follows:

The ATP content was detected using the ATP detection kit produced by Beyotime Biotechnology. Approximately 100-200 μL of a lysis buffer was added to every 20 mg of tibialis anterior (TA) tissue, and then homogenized. Sufficiently homogenization was performed to ensure that the tissue was completely lysed. After lysis, the mixture was centrifuged at 12,000 g for 5 minutes at 4° C., and the supernatant was taken and used for subsequent determination. Preparation of standard curve determination: the reagents to be used were thawed on an ice bath, and the ATP standard solution was diluted with the ATP detection lysis buffer to an appropriate concentration gradient. The specific concentration was to be determined according to the concentration of ATP in the tissue samples. Preparation of ATP detection working solution: an appropriate amount of the ATP detection working solution was prepared according to the proportion that 100 μL of the ATP detection working solution was needed for each sample or standard. The reagents to be used were thawed on an ice bath. An appropriate amount of the ATP detection reagent was taken, and the above detection reagent was diluted with the diluent at a ratio of 1:9. 100 μL of the ATP detection working solution was added into the detection tube. The detection tube was placed at room temperature for 3-5 min, so that all the background ATP was consumed completely, thus reducing the background. 20 μL of the sample or standard was added into the detection tube and mixed rapidly with a pipette (micropipette). After an interval of at least 2 seconds, the RLU value was measured with a chemiluminescence instrument. The corresponding ATP content was converted according to the standard curve. The results are shown in Table 4.

TABLE 4
Effect of the male flower extract of Eucommia ulmoides
Oliver on changes in the ATP content in the tibialis
anterior of naturally aging mice (n = 8)

| Group | ATP content (nM/g) |
|---|---|
| Young group | 815.6 ± 12.4*** |
| Elderly control group | 486.7 ± 10.9 |
| Example 1 | 512.9 ± 8.6^{###} |
| Low-dose group | |
| Example 1 | 554.8 ± 10.2^{###} |
| Medium-dose group | |
| Example 1 | 577.7 ± 11.5^{###} |
| High-dose group | |

Note:
*indicates the comparison between the elderly control group and the younger group, and
***represents P < 0.001; and
^{#}indicates the comparison between the low-, medium-, and high-dose groups of Example 1 and the elderly control group, and
^{###}represents P < 0.001.

Mitochondria are the main sites of oxidative phosphorylation and synthesis of adenosine triphosphate (ATP) within cells, providing chemical energy for cell activities. To some extent, the content of ATP in skeletal muscles reflects the normal level of mitochondrial function in this tissue. As shown in Table 4, under the effect of aging and a high-fat diet, the content of ATP in the tibialis anterior of the elderly mice decreased, and the intervention with the male flower extract of Eucommia ulmoides Oliver effectively prevented the decrease of ATP, indicating that the male flower extract of Eucommia ulmoides Oliver can effectively prevent the decline of skeletal muscle mitochondrial function in the aging process.

### Activity Detection Example 6

Study on the activity of the male flower extract of Eucommia ulmoides Oliver fed to 5-6-month-old ICR elderly mice induced by d-galactose to relieve muscle hypofunction. The specific method was as follows:

The 5-6-month-old ICR male young mice purchased from the Animal Research Center of East China Normal University were housed in the specific pathogen-free animal facility of Zhejiang Chinese Medical University. During this period, the mice were allowed to eat and drink freely. The dark/light cycle in the animal facility was 12 hours, and the temperature and humidity were maintained at 23±2° C. and 50%±5%, respectively. All the procedures and protocols in this experiment were approved by the Animal Ethics Committee of Zhejiang Chinese Medical University according to the standards.

After two weeks of adaptation, the mice were divided into the following five groups, including the control group, the model group, and the low-, medium-, and high-dose intervention groups of the male flower extract of Eucommia ulmoides Oliver after modeling (administered by gavage at 25, 50 and 100 mg/kg/d, respectively), with 8 mice in each group. The control group was gavaged with an equal volume of normal saline. All the mice were fed with standard feed. The modeling method was to perform aging modeling induced by d-galactose, and d-galactose (60 mg/kg body weight/day, 0.5 mL intraperitoneally) was administered for 1.5 months. After the male flower extract of Eucommia ulmoides Oliver intervened in the above aging mouse models for 3 months, the determination of various indicators in Activity Detection Examples 4 and 5 was performed. The results are shown in Tables 5-8.

TABLE 5
Effect of the male flower extract of Eucommia ulmoides Oliver on changes
in skeletal muscle mass of aging mice induced by d-galactose (n = 8)

| Group | GA (mg) | SOL (mg) | TA (mg) | EDL (mg) |
|---|---|---|---|---|
| Control group | 220.4 ± 4.1*** | 10.0 ± 0.8*** | 70.8 ± 4.3*** | 14.3 ± 0.3*** |
| Model group | 180.5 ± 2.3 | 4.2 ± 0.7 | 55.6 ± 1.7 | 12.5 ± 0.2 |
| Example 1 | 185.7 ± 2.1^{###} | 5.8 ± 0.5^{###} | 58.4 ± 1.2^{##} | 12.6 ± 0.4 |
| Low-dose | | | | |
| group | | | | |
| Example 1 | 191.5 ± 2.2^{###} | 6.2 ± 0.4^{###} | 63.3 ± 2.5^{###} | 12.9 ± 0.3 |
| Medium-dose | | | | |
| group | | | | |
| Example 1 | 192.0 ± 1.8^{###} | 6.3 ± 0.4^{###} | 64.2 ± 1.7^{###} | 13.0 ± 0.5^{#} |
| High-dose | | | | |
| group | | | | |

Note:
*indicates the comparison between the elderly control group and the young group,
*represents P < 0.05,
**represents P < 0.01, and
***represents P < 0.001;
^{#}indicates the comparison between the low-, medium-, and high-dose groups of Example 1 and the elderly control group,
^{#}represents P < 0.05,
^{##}represents P < 0.01, and
^{###}represents P < 0.001;
and the same applies below.

TABLE 6
Effect of the male flower extract of Eucommia ulmoides
Oliver on changes in forelimb grip strength of aging
mice induced by d-galactose (n = 8)

| | Average maximum | Average maximum forelimb |
|---|---|---|
| | forelimb grip | grip strength (N)-after |
| Group | strength (N)-initial | three months |
| Control group | 5.1 ± 0.8*** | 5.8 ± 1.0*** |
| Model group | 2.1 ± 0.3 | 2.8 ± 0.4 |
| Example 1 | 2.2 ± 0.2 | 3.6 ± 0.7^{#} |
| Low-dose | | |
| group | | |
| Example 1 | 2.3 ± 0.2 | 4.5 ± 0.5^{###} |
| Medium-dose | | |
| group | | |
| Example 1 | 2.1 ± 0.4 | 4.6 ± 0.1^{###} |
| High-dose | | |
| group | | |

TABLE 7
Effect of the male flower extract of Eucommia ulmoides
Oliver on changes in exhaustive running time of
aging mice induced by d-galactose (n = 8).

| | | Average exhaustive |
|---|---|---|
| | Average exhaustive running | running time (min)-after |
| Group | time (min)-initial | three months |
| Control group | 92.3 ± 1.7*** | 95.4 ± 2.1*** |
| Model group | 66.2 ± 2.7 | 68.2 ± 1.4 |
| Example 1 | 67.1 ± 1.5 | 70.8 ± 1.1^{##} |
| Low-dose | | |
| group | | |
| Example 1 | 69.4 ± 1.4 | 80.0 ± 0.8^{###} |
| Medium-dose | | |
| group | | |
| Example 1 | 67.4 ± 2.1 | 81.5 ± 1.2^{###} |
| High-dose | | |
| group | | |

TABLE 8
Effect of the male flower extract of Eucommia ulmoides
Oliver on changes in the ATP content in the tibialis anterior
of aging mice induced by d-galactose (n = 8)

| Group | ATP content (nM/g) |
|---|---|
| Control group | 965.3 ± 11.7*** |
| Model group | 516.4 ± 8.9 |
| Example 1 | 533.5 ± 9.6^{##} |
| Low-dose group | |
| Example 1 | 634.8 ± 10.1^{###} |
| Medium-dose group | |
| Example 1 | 678.7 ± 10.5^{###} |
| High-dose group | |

Experimental data in Tables 5-8 showed that the reduction in skeletal muscle mass induced by d-galactose was mainly manifested in the gastrocnemius (GA), tibialis anterior (TA) and soleus (SOL), among which the reduction in soleus mass was different from that in the case of natural aging. The male flower extract of Eucommia ulmoides Oliver prepared in Example 1 can effectively reduce the losses of the gastrocnemius (GA), tibialis anterior (TA) and soleus (SOL). Regarding skeletal muscle function, the low-, medium-, and high-dose interventions of the male flower extract of Eucommia ulmoides Oliver can improve the decreases in forelimb grip strength and exhaustive running time induced by d-galactose. Similarly, the low-, medium-, and high-dose interventions of the male flower extract of Eucommia ulmoides Oliver improved the decline in the ATP content in the tibialis anterior of the mice induced by d-galactose. Therefore, the male flower extract of Eucommia ulmoides Oliver had the efficacy of relieving aging-related muscle hypofunction.

### Application Example 1 Preparation of a Solid Beverage

The male flower extract of Eucommia ulmoides Oliver 34.9% (prepared in Example 1), xylitol 40%, fructooligosaccharide 5%, inulin 5%, maltodextrin 15%, and citric acid 0.1%.

The preparation method was performed according to the above formula, and the following steps were performed in sequence:

(1) weighing the materials according to the proportion of the above formula, mixing and stirring evenly, and drying until the water content was ≤5% to obtain a solid beverage product of the male flower extract of Eucommia ulmoides Oliver. All the other materials were commercially available products with qualified quality.

(2) quantitatively packaging the evenly stirred materials, and then performing microwave sterilization. The frequency of the microwave sterilization was 2000 MHz. In the sterilization process, the temperature of the beverage was controlled at 65° C. The sterilization time was determined according to the flow rate of the beverage and microwave power. After sterilization, both the total number of colonies and the yeast content in the beverage did not exceed the provisions of the national standards.

Finally, it is also to be noted that what has been listed above are only specific embodiments of the present invention. Obviously, the present invention is not limited to the above embodiments, and many modifications can be made. All modifications that can be directly derived from or associated with the disclosure of the present invention by one of ordinary skill in the art shall be regarded as falling within the scope of protection of the present invention.

1. A method of preparing a food or a medicament for preventing or alleviating aging-related muscle hypofunction, comprising the steps of:
(a) adding a dried product of a male flower of Eucommia ulmoides Oliver into an aqueous solution of an organic solvent with a volume ratio of 65%-80% at a solid-liquid ratio of 1 g:20 mL-60 mL, wherein the organic solvent is ethanol, methanol or n-butanol, extracting at a condition of 20-65° C., and separating to obtain an extracted liquid;
(b) removing the organic solvent in the extracted liquid, then separating to obtain an extract liquid after adding water for full reconstitution, and drying to obtain a male flower extract of Eucommia ulmoides Oliver; and
(c) combining the male flower extract of Eucommia ulmoides Oliver with a food-acceptable excipient or pharmaceutically acceptable excipient;
wherein the food comprises a human food and a pet food, and an active ingredient in the male flower extract of Eucommia ulmoides Oliver comprises iridoids;
wherein the iridoids comprise aucubin, geniposide, geniposidic acid and asperuloside.

2. (canceled)

3. The method according to claim 1, wherein the preventing or alleviating aging-related muscle hypofunction comprises improving skeletal muscle mass, skeletal muscle strength, mitochondrial function and body motor ability.

4. The method according to claim 1, wherein the male flower extract of Eucommia ulmoides Oliver improves the mitochondrial function to delay muscle hypofunction by activating mitophagy activity of muscle cells, promoting adenosine triphosphate (ATP) synthesis and/or maintaining normal mitochondrial morphology.

5.-6. (canceled)

7. The method according to claim 1, wherein in step (a), a water content of the dried product of the male flower of Eucommia ulmoides Oliver is ≤10%, and a solid-liquid ratio is 1 g:40 mL.

8. The method according to claim 1, wherein in step (a), a temperature of the extracting is 45-60° C., and time of the extracting is 20-30 hours.

9. The method according to claim 1, wherein in step (a), an extraction process is combined with ultrasonic treatment, and ultrasonic conditions are as follows: an ultrasonic frequency is 200-500 kHz, an ultrasonic intensity 5-15 W/cm², time is 1.5-3.0 hours, and a solution for the extracting is left to stand still after the ultrasonic treatment is completed.

10. (canceled)
